(19) United States
(12) Patent Application Publication
(10) Pub. No.: US 2002/0123503 A1
(43) Pub. Date: Sep. 5, 2002

(54) Cabergoline pharmaceutical compositions and methods of use thereof

(76) Inventors: Malcolm Ross, Tel-Aviv (IL); David Caron, Nordia (IL); Hava Schneider, Kfar Saba (IL); Bosmat Rousso, Petach Tikva (IL); Irena Ulanenko, Netanya (IL)

Correspondence Address: Mark S. Cohen, EITAN, PEARL, LATZER, & COHEN-ZEDEK, ONE CRYSTAL DRIVE, SUITE 210, 2011 CRYSTAL DRIVE, ARLINGTON, VA, 22202-3709, US

(21) Appl. No.: 09/740,955

(22) Filed: Dec. 21, 2000

(51) Int. Cl. A61K 31/44; A01N 43/42

(52) U.S. Cl. 514/285; 424/449

(57) ABSTRACT

The invention relates to pharmaceutical compositions of cabergoline and to processes for the preparation and methods of use thereof.

### FIELD OF THE INVENTION

The invention relates to pharmaceutical compositions, and processes for the preparation thereof, comprising cabergoline and derivatives thereof.

### BACKGROUND OF THE INVENTION

Cabergoline is a dopamine receptor antagonist and has been reported as useful for the treatment of a wide range of neurological and endocrinological conditions, including Parkinson's disease and other movement disorders, galactorrhea and other hyperprolactinemias, glaucoma, insomnia and parasomnias, and impotence and infertility, among others. Cabergoline is described in U.S. Pat. No. 4,526,892 which is herein incorporated by reference.

Cabergoline is formulated as an oral dosage form such as a tablet or capsule, however the formulation of this material presents certain unique difficulties. The therapeutic dose required for this drug may be as low as 500 microgram and, thus, the active ergoline needs to be extensively diluted with normal tablet excipients in order to provide a dosage weight that is convenient to the patient. In solid dose formulations it is conventionally accepted that when an extensive dilution of this nature is required it is necessary or preferred to carry out a wet granulation process [Bandelin in Compressed tablets by wet granulation p149-Pharmaceutical Dosage Forms: Tablets Vol 1 2^{nd} ed. Marcel Dekker, 1989]. This is because it is extremely difficult to ensure adequate mixing to provide a homogenous product, thereby guaranteeing the correct amount of active material in each unit dosage form. Such uniformity is required by regulatory authorities such as the FDA [Requirements for Uniformity of Final Blend—Draft Guidelines]. Also, conventional wet granulation processes to make cabergoline pharmaceutical compositions are prohibited by the extremely unstable nature of cabergoline, particularly in the presence of moisture.

### SUMMARY OF THE INVENTION

According to the present invention, the invention provides a composition comprising: 1) a pharmaceutically acceptable excipient, or excipients, and an ergoline cabergoline, whereby said excipient and said ergoline are combined by a wet granulation process using a substantially non-queous granulating fluid, so as to preserve the stability of said ergoline and permit homogenous dispersion of said ergoline in said composition; and, 2) a pharmaceutically acceptable carrier or diluent.

In another embodiment, the present invention provides a stable, homogeneous granule comprising the composition according to the invention.

In another embodiment, the present invention provides a pharmaceutical vehicle suitable for enteral administration.

The invention provides a process for producing an ergoline cabergoline composition, comprising the steps of: 1) combining an amount of cabergoline, a non-aqueous granulating fluid, and an amount of a pharmaceutically acceptable excipient to produce a mixture; 2) blending the mixture to form a wet granulate; and, 3) drying said granulate to yield said composition.

In another embodiment of the present invention is a method for the manufacture of a pharmaceutically acceptable vehicle for enteral administration, comprising the process of producing an ergoline cabergoline composition, comprising the steps of: 1) combining an amount of cabergoline a non-aqueous granulating fluid, and an amount of a pharmaceutically acceptable excipient to produce a mixture; 2) blending the mixture to form a wet granulate; and, 3) drying said wet granulate to yield said composition; 4) incorporating said composition into a pharmaceutically acceptable vehicle.

The present invention provides a method for the treatment of a subject predisposed or afflicted with a medical disorder, comprising the step of administering to a subject a therapeutically effective amount of a pharmaceutical vehicle comprising 1) a pharmaceutically acceptable excipient, or excipients, and an ergoline cabergoline, whereby said excipient and said ergoline are combined by a wet granulation process using a substantially non-queous granulating fluid, so as to preserve the stability of said ergoline and permit homogenous dispersion of said ergoline in said composition; and, 2) a pharmaceutically acceptable carrier or diluent, thereby preventing or treating the medical disorder.

### DETAILED DESCRIPTION OF THE INVENTION

The invention disclosed herein provides a physically and chemically stable, oral dosage composition form of cabergoline, or a pharmaceutically acceptable salt thereof, for administration to subjects In need thereof, and which dosage form substantially overcomes the deficiencies associated with the prior art. The invention disclosed herein provides a process for the incorporation of cabergoline into a stable and homogeneous dosage form, including but not only, granules, powders, tablets, capsules, and lozenges. Based on the compositions disclosed herein, the present invention further provides for methods of treatment of disease based on administration of the stable and homogeneous dosage forms of the ergoline disclosed herein.

It is well known to those skilled in the art that wet granulation is a preferred procedure to ensure content uniformity in the manufacture of low dose products. However wet granulation is not possible where the active ingredient is highly unstable as is the case with cabergoline and indeed using normal wet granulation techniques cabergoline did degrade.

Surprisingly, it has been found that a homogenous product could be prepared by a wet granulation process without excessive degradation by using a non-aqueous granulation process. Preferably, the process comprises dissolving the cabergoline, or a pharmaceutically acceptable salt thereof, in an alcohol such as ethanol or isopropanol and adding the required amount to selected excipients in a high shear mixer. The granulate is then dried, mixed with a lubricant and either filled into capsules or compressed. The product of such process affords a granulation with good quality attributes that could be compressed to afford a tablet of required hardness and friability.

In addition, it has been found that although such tablets were found to possess good chemical stability, this chemical stability could be further improved by the addition of an organic acid during the granulation step. Thus, the present invention provides a process for the preparation of a stable, homogenous granulate containing cabergoline and one or more pharmaceutical excipients suitable for the production of solid unit dosage forms. The process comprises a wet granulation process whereby the granulate is made by dissolving the cabergoline, or a pharmaceutically acceptable salt thereof, in a non-aqueous or essentially non-aqueous solvent as part of the granulating fluid. In the process, addition of an organic acid at one or more of the stages of the granulating process results in further enhanced stability of the cabergoline ingredient of the pharmaceutical composition, and granules thereof.

Cabergoline is described in U.S. Pat. No. 4,526,892, incorporated herein by reference, as a novel ergoline derivative and a process for its preparation is provided therein. The chemical name for the compound is 1((6-allylergolin-8.beta,-yl)-carbony.)-1-(3-(dimethylamino)propyl)-3ethylurea or ((8β)-N-[3-(dimethylamino)prop)]-N-[(ethylamino)carbonyl]-6-(2-propenyl) ergoline-8-carboxamide). Carbergoline is the active ingredient in DOSTINEX.RTM. and CABASER.RTM. tablets sold in the United States, Europe and Latin America as a treatment for hyperprolactinemic disorders and/or as a treatment for Parkinson's disease. Cabergoline is primarily known as a dopamine receptor agonist and has been reported as useful for the treatment for a wide range of neurological and endocrinological disorders, as well as other dopamine agonist disorders.

Cabergoline is a unique dopamine agonist with unusual properties, including differential effects towards a range of dopamine receptors. Unlike other D2 receptor family agonists, cabergoline is also pharmacologically unique in being a full D2 agonist With partial D1 activity.

This invention provides a composition for use in producing a pharmaceutical vehicle of cabergoline, thereby allowing for the formation of more stable dosage forms. Thus, for example, as described herein, a substantially more stable tablet form of the drug may be produced. The property of enhanced stability includes a harder tablet that is less friable and more durable upon handling than the conventional dosage form. This property includes as well a greater shelf life and more reliable means for administration of a pre-determined dose of the drug.

In one embodiment, the invention provides a pharmaceutically acceptable composition comprising an ergoline drug for treating neurological or endocrine disorders in a subject in need of administration thereof, and which pharmaceutically acceptable composition can be delivered from a dosage form manufactured to a high degree of physical and chemical stability, and homogeneity, thus ensuring reliable dosing of the drug.

In another embodiment, the present invention provides a dosage form adapted for oral administration of a dopamine agonist, which dosage form comprises cabergoline, and which pharmaceutically acceptable composition can be delivered from a dosage form manufactured to a high degree of physical and chemical stability, and homogeneity, thus ensuring reliable dosing of the drug.

In another embodiment, the present invention provides a dosage form for administering the ergoline carbergoline over a prolonged period of time for treating neurological or endocrine disorders, in particular dopamine agonist responsive disorders.

In another embodiment, the present invention provides a method for treating a neurological or endocrinological disorder by orally administering a pharmaceutically acceptable composition of the ergoline cabergoline in a rate controlled dose per unit time to a subject, i.e. a warm-blooded animal, in need of anti-Parkinson or anti-prolactin therapy and which pharmaceutically acceptable composition thereof can be delivered from a dosage form manufactured to a high degree of physical and chemical stability, and homogeneity, thus ensuring reliable dosing of the drug.

In another embodiment, the present invention provides a process for the manufacture of an oral solid dosage form of cabergoline, which is economical to manufacture and reliably reproducible.

In another embodiment, the present invention provides an oral solid dosage form for cabergoline, which maintains its integrity during storage, while possessing suitable disintegration and dissolution properties, e.g., to gastrointestinal fluid, when administered in therapeutic doses.

In another embodiment, the present invention provides an improved oral dosage form of cabergoline which is physically and chemically stable and which has improved hardness and reduced friability relative to “off-the-shelf” commercially available preparations.

In another embodiment, the present invention provides a solid dosage form which Includes one or more active ingredients for the treatment of a subject afflicted with a neurological disorder, an endocrinological disorder, a dopamine agonist responsive disorder, or a combination thereof.

In another embodiment, the present invention provides an improved oral dosage form of cabergoline which is more readily administered, e.g., easier to handle and to swallow, thus allowing for safer dosing of the active agent and therefore an improved regimen of therapy by oral cabergoline administration.

In another embodiment, the present invention provides an improved oral dosage form of cabergoline which is more readily administered, e.g., easier to handle and to swallow, thus allowing for better patient compliance and therefore an improved regimen of therapy by oral cabergoline administration.

According to the invention, the use of a non-aqueous wet granulation process for the manufacture of a composition comprising cabergoline, and for granules and pharmaceutical vehicles thereof, prevents segregation of the cabergoline from among other ingredients of the composition. Thus, the use of the non-aqueous wet granulation process for combining cabergoline, allows the homogeneous dispersion of cabergoline in the resultant pharmaceutically acceptable composition. Preferably the process comprises dissolving the cabergoline in a stable alcohol such as ethanol or isopropanol and adding the required amount to selected excipients in a high shear mixer. The granulate is then dried, mixed with a lubricant and either filled into capsules or compressed. The product of such process affords a granulation with good quality attributes that could be compressed to afford a tablet of required hardness and friability. In addition, we found that although such tablets were found to possess good chemical stability, this chemical stability could be further improved by the addition of an organic acid during the granulation step.

Suitable materials for a composition of cabergoline according to the present invention include pharmaceutically acceptable excipients, including, but not only, the following:

i) Binders, such as cellulose and its derivatives, e.g. ethyl cellulose hydroxypropylmethyl cellulose, hydroxypropylcellulose, hydroxyethyl cellulose, sodium carboxymethyl cellulose, starches, polyvinyl pyrrolidone, natural gums, corn syrup, polysaccharides (including acacia, tragacanth, guar, and alginates), gelatin, or a combination thereof;

ii) Glidants, such as talc, fumed silica, or a combination thereof;

iii) Lubricants such as magnesium stearate, calcium stearate, aluminum stearate, stearic acid, calcium oleate, talc, mineral oil, waxes, glyceryl behenate, potassium stearyl fumarate, sodium stearyl fumarate, hydrogenated vegetable oils, or a combination thereof. Such lubricants are commonly included in the final tabletted product in amounts of less than 1% by weight.

iv) Diluents, such as lactose, cellulose, starch or calcium phosphate or a combination thereof.

The term “a granulate” is intended to mean the granulate obtainable by using the wet granulation method and has the general meaning as disclosed in e.g. Remington; The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997. The term “a granules” refers herein to the one or more particulate structural components comprising a granulate, in either a dry state, a semi-dry state, a wet state, or a combination thereof. A granule or granules may have any suitable size, depending on the carriers and/or equipment used and the preparation of granules with a particular size and structure is within the technical knowledge of the skilled person.

The term “a wet granulation method” represents a conventional way of making a granule, granules, or a granulate and is disclosed in e.g. Remington: The Science and Practice of Pharmacy, 19^{th} Edition (1995) and/or in Handbook of Pharmaceutical Granulation Technology. Chapter 7, “Drugs and the Pharmaceutical Sciences”, vol. 81. 1997. The wet method usually comprises the steps of weighing, mixing, granulation, screening the damp mass, and drying. The wet method may further comprise an additional step or steps of dry screening, lubrication, compression, or a combination thereof.

The terms “granulation” and “granulation of the mixture” are intended to have the usual meaning, as disclosed in Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997; and Include one or more steps of dry blending and wet massing performed prior to or after a granulation step.

The term “drying the mixture” is intended to have its usual meaning, as disclosed in Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997; and comprises drying the granulation mixture in a conventional manner either inside or outside mixing means, such as but not only a granulator or a high shear mixer, by placing the moist granulation mixture, such as but not limited to, drying cabinets with circulating air current and thermostatic heat control.

The term “processing the granulate” is intended to mean the further conventional processing of the granulate into an oral solid dosage formulation as disclosed in Remington: The Science and Practice of Pharmacy, 19^{th} Edition (1995) and/or in Handbook of Pharmaceutical Granulation Technology, Chapter 7, “Drugs and the Pharmaceutical Sciences”, vol. 81, 1997; and comprises, but is not limited to, reducing the granules or granulate to a particular a particular size, lubrication, and compressing into tablets, lozenges, powders, or filling into gelatin capsules.

The terms “pharmaceutically acceptable” and “or a salt thereof” represent forms of an ingredient that are physiologically acceptable for pharmaceutical use, including pharmaceutically acceptable salts thereof,

The terms “an oral solid dosage formulation” or “an oral solid dosage from” or “a pharmaceutically acceptable vehicle suitable for enteral administration” are intended to mean such solid dosage formulations as disclosed in Remington: The Science and Practice of Pharmacy. 19^{th} Edition (1995) and/or in Handbook of Pharmaceutical Granulation Technology, Chapter 7, “Drugs and the Pharmaceutical Sciences”, vol. 81, 1997; and comprises, but is not limited to, tablets including comestibles, capsules, pills, lozenges, troches, cachets, and pellets.

According to another embodiment of process of the Invention, the ergoline cabergoline is dissolved in a non-aqueous granulating fluid. This mixture is then used to granulate a pharmaceutically acceptable excipient, blending for sufficient time to allow granulation to occur, preferably in the complete absence of water. The granulating fluid may be added, for example, by drops or spraying. The resulting mixture is then dried to form a stable composition of the ergoline homogeneously combined with an excipient. Suitably dried, the composition consists of stable, homogeneous granules suitable for further incorporation into a solid dosage form. According to another embodiment of the process, a pharmaceutically acceptable acid is added to the granulating fluid, either prior to or following the addition of the excipient.

According to another embodiment of the process of the invention, the ergoline and organic acid are combined prior to addition of the granulating fluid.

According to another embodiment of the process of the invention, the ergoline, the excipient, and the organic acid are combined prior to addition of the granulating fluid.

According to another embodiment of the process of the invention, a second pharmacologically active ingredient is added to the composition, at any stage of the process, either prior to and/or following the step of drying, to produce a composition comprising an ergoline and a second pharmacologically active ingredient.

As used herein, the term “non-aqueous granulating fluid” refers to any fluid composition which is capable of dispersing or dissolving either cabergoline, or one or more excipients of interest, and which is sufficiently devoid of water so as to maintain the chemical integrity of cabergoline. Representative examples include alcohols, such as ethanol or isopropanol, ethers, ketones, hydrocarbons, halogenated hydrocarbons, and other organic solvents. The relative hardness of tablets produced according to the process of the invention exceeds that of “off-the-shelf” commercially available cabergoline preparations, as judged by measuring both indexes of friability and hardness. As represented by the Examples below, tables produced according to the invention have a friability of less than 0.8% and a hardness of at least 3 kilopond and as high as 20 kilopond. Friability was measured according to the method of the United States Pharmacopia and hardness was measured by crushing using commercial tablet hardness testers.

In a preferred embodiment of the present pharmaceutical composition, especially wherein a granulate comprising the composition is compressed into tablet form, suitable ingredients are: cabergoline, microcrystalline cellulose, citric acid anhydrous, CrosCarmellose, magnesium stearate, and, as granulating fluid, isopropyl alcohol, anhydrous.

The present pharmaceutical composition is prepared by substantially non-aqueous wet granulation techniques. Thus, the Invention provides a wet granulation process for the preparation of a pharnaceutical composition according to the invention comprising: a process for producing a composition of cabergoline, whereby said process comprises the steps of: combining an amount of a pharmaceutically acceptable excipient and cabergoline; adding an amount of a substantially non-aqueous granulating fluid to form a wet granulate; blending; and, drying to yield said composition.

In another embodiment, the invention provides a process for producing a composition of cabergoline, whereby said process comprises the steps of: combining an amount of a pharmaceutically acceptable excipient and cabergoline; adding an amount of an organic acid; adding an amount of a substantially non-aqueous granulating fluid to form a wet granulate; blending; and, drying to yield said composition.

In the foregoing embodiments, the steps of the process may be performed in an order which allows one skilled in the art to obtain the composition, granules thereof, and pharmaceutically acceptable vehicles thereof, in expeditious manner which does not diminish from the desired properties of the final product. Thus, for example, the drug and the excipient may first be dry mixed, together With any additional excipients, as required. The mixed powders are then granulated by wetting with a non-aqueous granulating fluid. In another embodiment, the drug or excipients are first combined with the granulating fluid and then further excipient and/or drug is added to form a blend. Thus the methods of the invention for preparing stable, homogeneous cabergoline compositions, granules, and tablets thereof, comprise steps that may be performed in a variety of sequences in order to achieve the desired product. Determining the order of the steps for implementing the process of the invention may be readily appreciated by one skilled in the art of dispensary.

The granulating fluid may contain, as desired, an acid, preferably an organic acid. Similarly, the acid may be added in dry form to the other ingredients, or first dissolved In the granulating fluid prior to addition of the other ingredients to form a blend. The addition of further excipients, including binders, lubricants, carriers, disintegrants, diluents, anti-adhesives, or a combination thereof, may be similarly performed.

Thus, in one embodiment, the invention provides a process for producing a composition comprising: 1) a pharmaceutically acceptable excipient and the ergoline cabergoline, or a derivative thereof, whereby said excipient and said ergoline are combined by a wet granulation process using a substantially non-aqueous granulating fluid, so as to preserve the stability of said ergoline and permit homogenous dispersion of said ergoline in said composition; and, 2) a pharmaceutically acceptable carrier or diluent, whereby said process comprises the steps of: combining an amount of cabergoline, a non-aqueous, granulating fluid, and an amount of a pharmaceutically acceptable excipient; blending to form a wet granulate; and, drying to yield said composition. In a preferred embodiment of the process, said excipient is selected from the group consisting of binders, carriers, disintegrants, diluents, lubricants, glidants, or a combination thereof. In another embodiment, said process further comprises the step of adding a pharmaceutically acceptable acid, preferably an organic acid, more preferably a carboxylic acid, a dicarboxylic acid, an amino acid, or a combination thereof. In another embodiment of said process, said non-aqueous granulating fluid is an organic solvent, preferably an anhydrous alcohol, more preferably an alcohol selected from the group consisting of methanol, ethanol, isopropyl alcohol, benzyl alcohol, polyethylene glycol, propylene glycol, or a combination thereof.

In another embodiment, the invention provides a process for producing a composition comprising: 1) a pharmaceutically acceptable excipient and the ergoline cabergoline, or a derivative thereof, whereby said excipient and said ergoline are combined by a wet granulation process using a substantially non-aqueous granulating fluid, so as to preserve the stability of said ergoline and permit homogenous dispersion of said ergoline in said composition; and, 2) a pharmaceutically acceptable carrier, or diluent, whereby said process comprises the steps of: combining an amount of cabergoline, a non-aqueous granulating fluid, an amount of a pharmaceutically acceptable excipient, and an amount of a pharmaceutically acceptable acid; blending to form a wet granulate; and, drying to yield said composition.

In a preferred embodiment of the process, said exciplent is selected from the group consisting of binders, carriers, disintegrants, diluents, lubricants, glidants, or a combination thereof. In another embodiment, said process further comprises the step of adding a pharmaceutically acceptable acid, preferably an organic acid, more preferably a carboxylic acid, a dicarboxylic acid, an amino acid, or a combination thereof.

In another embodiment of said process, said non-aqueous granulating fluid is an organic solvent, preferably an anhydrous alcohol, more preferably an alcohol selected from the group consisting of methanol, ethanol, isopropyl alcohol, benzyl alcohol, polyethylene glycol, propylene glycol, or a combination thereof.

According to preferred embodiments of the invention, a pharmaceutically acceptable acid is an organic acid, preferably an amino acid, a carboxylic acid, or a dicarboxylic acid. Examples of preferred organic acids are: citric, tartaric, maleic, glycine, lysine, methanesulfonic, leucine, or a combination thereof.

According to preferred embodiments of the invention, the granulating fluid is comprised of one or more organic solvents, preferably alcohols. Examples of preferred alcohols are: methanol, ethanol, isopropyl alcohol, benzyl alcohol, polyethylene glycol, propylene glycol, or a combination thereof.

A tableting composition is preferably produced from the granulated mass or granules obtained directly from the dried granulate, or from an alcoholized granulate, by subjoinment with a tableting binder such as sodium carboxymethyl cellulose, a lubricant such as magnesium stearate, or disintegrants such as croscarmellose sodium. There are many other pharmaceutically acceptable tableting binders, lubricants and disintegrants well known in the pharmaceutical arts which are usable in the production of the tablets of the present invention.

Such tableting composition is then fed to a multi-station tablet press for compression into tablets. The tablets preferably have a friability of not less than 0.8% and a hardness of 3 kilopond, and more preferably between 6 and 20 kilopond, The tablets of the invention show satisfactory hardness and friability and they comply with the usual standards with respect to disintegration time.

Thus, the invention provides a composition comprising: 1) a pharmaceutically acceptable excipient and the ergoline cabergoline, whereby said excipient and said ergoline are combined by a wet granulation process using a substantially non-aqueous granulating fluid, so as to preserve the stability of said ergoline and permit homogenous dispersion of said ergoline in said composition; and, 2) a pharmaceutically acceptable carrier or diluent,

In one embodiment of the composition according to the invention, said non-aqueous granulating fluid is an organic solvent, preferably wholly or in part a pharmaceutically acceptable alcohol, more preferably an alcohol selected from the group consisting of methanol, ethanol, isopropyl alcohol, benzyl alcohol, polyethylene glycol, propylene glycol, or a combination thereof.

In another embodiment of the composition according to the invention, said wet granulation process comprises the step of adding a pharmaceutically acceptable acid, preferably a pharmaceutically acceptable carboxylic acid, dicarboxylic acid, amino acid, or a combination thereof, more preferably an organic acid selected from the group consisting of citric, tartaric, maleic, glycine, lysine, methanesulfonic, leucine, or a combination thereof.

In another embodiment of the composition according to the invention, said excipient is selected from the group consisting of carriers, binders, lubricants, diluents, disintegrants, or a combination thereof, wherein preferably said binder is selected from the group consisting of polyvinyl pyrrolidone, starch, starch derivatives, acacia, tragacanth, gelatin, cellulose derivatives, or a mixture thereof, preferably said pharmaceutically acceptable carrier is selected from the group consisting of sugars, dissacharides, polysaccharides, or a combination thereof, wherein preferably said pharmaceutically acceptable carrier is selected from the group consisting of calcium phosphate, cellulose, cellulose derivatives, lactose, or a combination thereof, and wherein said disintegrant is selected from the group consisting of sodium starch glycolate, pregelatinized starch, cross linked povidone, croscarmelose sodium, calcium pectinate, or a combination thereof.

In another embodiment of the composition according to the invention, said composition comprises an amount of cabergoline, ranging from 0.01% to 1% by dry weight of said composition.

In another embodiment of the composition according to the invention, said composition comprises an amount of cabergoline, ranging from 1% to 10% by dry weight of said composition.

In another embodiment, said composition is further coated with a pharmaceutically acceptable coating.

In another embodiment of the composition according to the invention, said coating is a sustained release coating which substantially prevents the release of said derivatives prior to arrival in the small intestine. By “sustained release” it is meant for purposes of the invention that the therapeutically active medicament is released from the formulation at a controlled rate such that therapeutically beneficial blood levels (but below toxic levels) of the medicament are maintained over an extended period of time, e.g., providing a 12 hour or a 24 hour dosage form. Sustained release formulations suitable for parenteral administrations, such as biodegradable polymer formulations, as known to one skilled in the art.

According to the present disclosure, the invention provides stable, homogeneous granules comprising the composition according to any one of the foregoing embodiments.

According to the present disclosure, the invention provides a pharmaceutical vehicle suitable for enteral administration comprising a composition according to any one of the foregoing embodiments of the invention, wherein said vehicle is selected from the group consisting of tablets, capsules, lozenges, powders, or granules.

The ergoline compositions of this invention may be administered as a purified, a substantially purified compound, or preferably as a pharmaceutical. The composition of the invention comprises the ergoline cabergoline, one or more pharmaceutically acceptable carriers, and optionally other therapeutic agents. Formulations of the ergoline(s) described herein encompass those directed to administration by various oral routes, including a sustained release formulation thereof. Preferably, for the purpose of implementing the methods of the invention, an oral dosage form is administered. The carrier(s) must be “acceptable” in that It has to be compatible with, and stabilizing of, the active ingredient(s) of the formulation, and not deleterious to the subject to be treated (“pharmaceutically acceptable”).

In another embodiment of the invention, said pharmaceutical vehicle comprises an amount of said cabergoline, or pharmaceutically acceptable derivative thereof, ranging from 0.01% to 1% by dry weight of said vehicle.

In another embodiment of the invention, said pharmaceutical vehicle comprises an amount of said cabergoline, or pharmaceutically acceptable derivative thereof, ranging from 1% to 10% by dry weight of said vehicle.

In general, the formulations for tablets or powders may be prepared by uniformly and intimately blending the active ingredient with finely divided solid carriers, and then, if necessary, as in the case of tablets, forming the product into the desired shape and size.

In another embodiment of the invention, said vehicle is a tablet having a friability of less than 0.8%.

In another embodiment of the invention, said vehicle is a tablet having a hardness of between 3 to 20 kilopond.

In another embodiment of the invention, said vehicle is a tablet having a hardness of between 6 to 12 kilopond.

The present pharmaceutical composition, together with processes for its preparation, will now be described by way of example only

### EXAMPLES

Tablets prepared in accordance With these Examples exhibited pharmaceutically acceptable properties with regard to stability, disintegration times and dissolution rates.

### EXAMPLE 1

Tablets were compressed from the following ingredients based on a non-aqueous wet granulation process:

| Material Name | mg/tab |
|---|---|
| Cabergoline | 0.5 |
| Microcrystalline Cellulose | 75.1 |
| Citric Acid Anhydrous | 1.2 |
| CrosCarmellose Sodium | 2.4 |
| Magnesium Stearate | 0.8 |

Cabergoline was dissolved in Isopropyl Alcohol and used to granulate the Microcrystalline Cellulose. Citric Acid Anhydrous was added and additional mixing was performed. Drying of the granulate was performed to give acceptable dry granules which were submitted to milling. The dried-milled granules were mixed with a disintegrant (CrosCarnellose) and with a lubricant (Magnesium Stearate). Finally, the lubricated blend was compressed to manufacture tablets. The uncoated tablets had a friability of less than 0.8% and a crushing strength of at least 6 kilopond.

### EXAMPLE 2

The procedure of Example 1 was followed except that the mixing order was changed:

| Material Name | mg/tab |
|---|---|
| Cabergoline | 0.5 |
| Microcrystalline Cellulose | 75.1 |
| Citric Acid Anhydrous | 1.2 |
| CrosCarmellose Sodium | 2.4 |
| Magnesium Stearate | 0.8 |

Cabergoline was dissolved in Isopropyl Alcohol and used to granulate a mixture consisting of Microcrystalline Cellulose and Citric Acid Anhydrous. Drying of the granulate was performed and the dried granulate was submitted to milling. The dried-milled granulate was mixed with the disintegrant (Croscarmellose) and with the lubricant (Magnesium Stearate). Finally, the lubricated blend was compressed to manufacture tablets. The uncoated tablets had a friability of less than 0.8% and a crushing strength of at least 6 kilopond.

### EXAMPLE 3

The procedure of Example 1 was followed except that the following composition was used.

| Material Name | mg/tab |
|---|---|
| Cabergoline | 0.5 |
| Microcrystalline Cellulose | 77.5 |
| Citric Acid Anbydrous | 1.2 |
| Magnesium Stearate | 0.8 |

Cabergoline was dissolved in Isopropyl Alcohol and used to granulate the Microcrystalline Cellulose. Citric Acid Anhydrous was added and additional mixing was performed. Drying of the granulate was performed to give an acceptable dry granulate which was submitted to milling. The dried-milled granulate was mixed with the lubricant (Magnesium Stearate). Finally, the lubricated blend was compressed to manufacture tablets. The uncoated tablets had a friability of less than 0.8% and a crushing strength of at least 6 kilopond.

### EXAMPLE 4

The procedure was based on Example 2 and the following composition was used:

| Material Name | mg/tab |
|---|---|
| Cabergoline | 0.5 |
| Lactose Anhydrous | 62.5 |
| Microcrystalline Cellulose | 16.0 |
| Magnesium Stearate | 1.0 |

Cabergoline was dissolved in Isopropyl Alcohol and used to granulate the mixture of Lactose Anhydrous and Microcrystalline Cellulose. Drying of the granulate was performed to give an acceptable dry granulate which was submitted to milling. The dried-milled granulate was mixed with the lubricant (Magnesium Stearate). Finally, the lubricated blend was compressed to manufacture tablets. The uncoated tablets had a friability of less than 0.8% and a crushing strength of at least 6 kilopond.

What we claim is:

1. A composition comprising: 1) a pharmaceutically acceptable excipient and an ergoline cabergoline, whereby said excipient and said ergoline are combined by a wet granulation process using a substantially non-aqueous granulating fluid, so as to preserve the stability of said ergoline and permit homogenous dispersion of said ergoline in said composition; and, 2) a pharmaceutically acceptable carrier or diluent.

2. The composition according to claim 1, wherein said non-aqueous granulating fluid is an organic solvent.

3. The composition according to claim 1, wherein said wet granulation process comprises the step of adding an acid.

4. The composition according to claim 1, wherein said wet granulation process comprises the step of adding an organic acid.

5. The composition according to claim 1, wherein said wet granulation process comprises the step of adding a carboxylic acid, a dicarboxylic acid, an amino acid, or a combination thereof.

6. The composition according to claim 4, wherein said organic acid is citric acid, tartaric acid, maleic acid, glycine, lysine, methanesulfonic acid, leucine, or a combination thereof.

7. The composition according to claim 1, wherein said granulating fluid is wholly or in part a pharmaceutically acceptable alcohol.

8. The composition according to claim 7, wherein said alcohol is methanol, ethanol, isopropyl alcohol, benzyl alcohol, polyethylene glycol, propylene glycol, or a combination thereof.

9. The composition according to claim 1, wherein said excipient is a carrier, a binder, a lubricant, a diluent, a disintegrant, or a combination thereof.

10. The composition according to claim 9, wherein said binder is polyvinyl pyrrolidone, starch, starch derivatives, acacia, tragacanth, gelatin, cellulose derivatives, or a mixture thereof.

11. The composition according to claim 9, wherein said carrier is a sugar, a dissacharide, a polysaccharide, or a combination thereof.

12. The composition according to claim 9, wherein said carrier is calcium phosphate, cellulose, a derivative of cellulose, lactose, or a combination thereof.

13. The composition according to claim 9, wherein said disintegrant is sodium starch glycolate, pregelatinized starch, crosslinked povidone, croscarmelose sodium, calcium pectinate, or a combination thereof.

14. The composition according to any one of claims 1 to 8, wherein said composition comprises an amount of cabergoline, ranging from 0.01% to 10% by dry weight of said composition.

15. The composition according to any one of claims 9 to 13, wherein said composition comprises an amount of cabergoline, ranging from 0.01% to 10% by dry weight of said composition.

16. The composition according to claim 1, wherein said composition is further coated with a pharmaceutically acceptable coating.

17. The composition according to claim 16, wherein said coating is a sustained release coating which substantially prevents the release of said derivatives prior to arrival in the small intestine.

18. A stable, homogeneous granule comprising the composition according to claim 1.

19. A stable, homogeneous granule comprising the composition according to claim 14.

20. A stable, homogeneous granule comprising the composition according to claim 15.

21. A pharmaceutical vehicle suitable for enteral administration comprising a composition according to claim 1, wherein said vehicle is selected from the group consisting of tablets, capsules, lozenges, powders, or granules.

22. The pharmaceutical vehicle according to claim 21, wherein the amount of said cabergoline, or pharmaceutically acceptable derivative thereof, ranges from 0.01% to 10% by dry weight of said vehicle.

23. The vehicle according to claim 22, wherein said vehicle is a tablet having a friability of less than 0.8%.

24. The vehicle according to claim 22, wherein said vehicle is a tablet having a hardness of between 3 to 20 kilopond.

25. The vehicle according to claim 22, wherein said vehicle is a tablet having a hardness of between 6 to 12 kilopond.

26. A process for producing a composition according to claim 1, whereby said process comprises the steps of:
combining an amount of cabergoline, a non-aqueous granulating fluid, and an amount of a pharmaceutically acceptable excipient;
blending to form a wet granulate; and,
drying to yield said composition.

27. The process according to claim 26, whereby said excipient is a binder, a carrier, a disintegrant, a diluent, a lubricant, a glidant, or a combination thereof.

28. The process according to claim 27, whereby said process further comprises the step of adding a pharmaceutically acceptable acid.

29. The process according to claim 28, whereby said acid is an organic acid.

30. The process according to claim 28, whereby said acid is a carboxylic acid, a dicarboxylic acid, an amino acid, or a combination thereof.

31. The process according to claim 26, whereby said non-aqueous granulating fluid is an organic solvent.

32. A method for the treatment of a subject predisposed or afflicted with a medical disorder, comprising the step of administering to a subject a therapeutically effective amount of a pharmaceutical vehicle according to any one of claims 21 to 25, thereby preventing or treating the medical disorder.

33. The method according to claim 32, wherein said medical disorder is a neurological disorder.

34. The method according to claim 32, wherein said medical disorder is an endocrinological disorder.
